(19) United States
(12) Patent Application Publication
(10) Pub. No.: US 2023/0010803 A1
(43) Pub. Date: Jan. 12, 2023

(54) TREATMENTS FOR DIFFUSE LARGE B-CELL LYMPHOMA

(71) Applicants: Janssen Pharmaceutica NV, Beerse (BE); The UNITED STATES OF AMERICA, as represented by the Secretary, Department of Health and Human, Bethesda, MD (US)

(72) Inventors: Sriram Balasubramanian, San Diego, CA (US); Brendan Hodkinson, Spring House, PA (US); Louise M. Staudt, Bethesda, MD (US); Wyndham H. Wilson, Menlo Park, CA (US); George Wright, Rockville, MD (US)

(21) Appl. No.: 17/854,211

(22) Filed: Jun. 30, 2022

Related U.S. Application Data
(60) Provisional application No. 63/216,777, filed on Jun. 30, 2021.

(51) Int. Cl. A61K 39/395; A61K 31/675; A61K 31/704; A61K 31/475; A61K 31/519; A61K 31/4985; A61P 35/00

(52) U.S. Cl. CPC A61K 39/3955; A61K 31/675; A61K 31/704; A61K 31/475; A61K 31/519; A61K 31/4985; A61P 35/00

(57) ABSTRACT

Disclosed are methods for treating specific genetic subtypes of diffuse large B cell lymphoma using a combination of a BTK inhibitor and R-CHOP chemotherapy. The methods produce favorable event-free survival and overall survival rates among subjects exhibiting the genetic subtypes.

### CROSS-REFERENCE TO RELATED APPLICATIONS

The present application claims the benefit of priority to U.S. Provisional Application No. 63/216,777, filed Jun. 30, 2021, the entire contents of which are incorporated herein by reference.

### TECHNICAL FIELD

The present disclosure pertains to targeted therapies for lymphoma patients, particularly members of certain genetic subgroups.

### BACKGROUND

The development of targeted therapy in diffuse large B cell lymphoma (DLBCL) is confounded by its extensive molecular heterogeneity. The first molecular classification, based on gene expression profiling, identified three groups—activated B cell-like (ABC), germinal center B cell-like (GCB) and unclassified DLBCL (Alizadeh A A, et al. Distinct types of diffuse large B-cell lymphoma identified by gene expression profiling. Nature 2000; 403:503-11)—that explained, in part, the differential response of DLBCL to chemotherapy (Rosenwald A, et al. The use of molecular profiling to predict survival after chemotherapy for diffuse large-B-cell lymphoma. N Engl J Med 2002; 346:1937-47). Genetic and functional studies demonstrated that chronic active B-cell receptor (BCR) signaling is a central oncogenic mechanism in ABC DLBCL that can be blocked by inhibitors of the kinase BTK.

This insight prompted a phase II study of the BTK inhibitor ibrutinib in relapsed/refractory DLBCL, which showed a differential response rate of 37% in ABC and 5% in GCB cases. This finding in turn prompted a phase III randomized evaluation of R-CHOP chemotherapy with or without ibrutinib (Phoenix Trial) in newly diagnosed patients with non-GCB DLBCL (Younes A, et al. Randomized Phase III Trial of Ibrutinib and Rituximab Plus Cyclophosphamide, Doxorubicin, Vincristine, and Prednisone in Non-Germinal Center B-Cell Diffuse Large B-Cell Lymphoma. J Clin Oncol 2019; 37:1285-95). Although the Phoenix study failed to meet its primary survival endpoint, a planned subset analysis in younger patients (<60 years) showed that event-free survival (EFS) and overall survival (OS) were 10.8% and 12.3% greater, respectively, in the ibrutinib arm compared to the placebo arm. By contrast, older patients on the ibrutinib arm experienced significantly more toxicity, received significantly less R-CHOP, and had a lower survival compared to the placebo arm, explaining the failure of the Phoenix trial to meet its overall primary endpoint.

### SUMMARY

Provided herein are methods for treating diffuse large B cell lymphoma comprising identifying a subject who (i) has diffuse large B cell lymphoma (DLBCL), (ii) exhibits an MCD genetic subtype or an N1 genetic subtype of DLBCL, and (iii) is 60 years of age or younger; and, treating the subject using a combination of a BTK inhibitor and R-CHOP chemotherapy.

Also disclosed are methods for treating diffuse large B cell lymphoma comprising identifying a subject who (i) has diffuse large B cell lymphoma (DLBCL), (ii) exhibits a genetic subtype characterized by at least a MYD88L265P mutation, a CD79B mutation, and either a PIM1 or a BTG1 mutation, or at least Notch1 truncation, and either a BCOR or an ID3 mutation, and (iii) is 60 years of age or younger; and, treating the subject using a combination of a BTK inhibitor and R-CHOP chemotherapy.

The present disclosure also provides methods for treating diffuse large B cell lymphoma comprising identifying a population of subjects who (i) have diffuse large B cell lymphoma (DLBCL), (ii) exhibit an MCD genetic subtype or an N1 genetic subtype of DLBCL, and (iii) are about 60 years of age or younger; and, treating a member of the population using a combination of a BTK inhibitor and R-CHOP chemotherapy.

### BRIEF DESCRIPTION OF THE DRAWINGS

FIGS. 1A-1C provide the results of an assessment of the genetic characteristics of DLBCL subtypes MCD, BN2, and N1.

FIGS. 2A-2C provide the results of an evaluation of RNA sequencing data to assess tumor phenotypes in each of the DLBCL subtypes MCD, BN2, and N1.

FIGS. 3A-3C illustrate the event-free survival and overall survival rates over time for subjects that were treated using an experimental protocol involving R-CHOP chemotherapy with or without ibrutinib co-therapy.

FIGS. 4A-4D depict the results of an analysis of publicly available tumor sequencing data for NOTCH1 mutations, in order to elucidate the molecular basis for ibrutinib efficacy in N1 patients.

### DETAILED DESCRIPTION OF ILLUSTRATIVE EMBODIMENTS

The presently disclosed inventive subject matter may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a treatment” is a reference to one or more of such treatments and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.” The phrase “at least about x” is intended to embrace both “about x” and “at least x”. It is also understood that where a parameter range is provided, all integers within that range, and tenths thereof, are also provided by the invention. For example, “2-5 hours” includes 2 hours, 2.1 hours, 2.2 hours, 2.3 hours etc. . . . up to 5 hours.

Since the initiation of the Phoenix trial, the molecular classification of DLBCL has been transformed by the analysis of genetic aberrations that include mutations, copy number alterations, and translocations. This new genetic taxonomy represents a refinement of the existing gene expression-based taxonomy: the ABC subgroup includes 4 genetic subtypes (MCD, BN2, N1, A53), GCB includes 4 genetic subtypes (EZB-MYC−, EZB-MYC+, ST2, BN2), and the Unclassified subgroup is largely comprised of the BN2 genetic subtype (Schmitz R, et al. Genetics and Pathogenesis of Diffuse Large B-Cell Lymphoma. N Engl J Med 2018; 378:1396-407; Wright G W, et al. A Probabilistic Classification Tool for Genetic Subtypes of Diffuse Large B Cell Lymphoma with Therapeutic Implications. Cancer Cell 2020; 37:551-68 e14). Using independent DLBCL cohorts and different analytic methods, three comprehensive genetic studies have converged on this consistent genetic subclassification of DLBCL tumors (Id.; see also Chapuy B, et al. Molecular subtypes of diffuse large B cell lymphoma are associated with distinct pathogenic mechanisms and outcomes. Nat Med 2018; 24:679-90; Lacy S E, et al. Targeted sequencing in DLBCL, molecular subtypes, and outcomes: a Haematological Malignancy Research Network report. Blood 2020; 135:1759-71). A probabilistic predictor termed LymphGen was recently developed to assign an individual DLBCL tumor to one or more of these genetic subtypes (Wright G W, et al.).

Importantly, these newly defined genetic subtypes differ with respect to pathogenesis, phenotypic properties and responses to therapy. A targeted therapeutic approach with respect to one or more genetic subtypes of DLBCL could yield better survival outcomes.

Molecular classification of DLBCL has therefore been transformed by the analysis of genetic aberrations that include mutations, copy number alterations, and translocations. This refined genetic classification of DLBCL is a promising framework in which to explain and ultimately predict responses to targeted therapies. For example, current evidence suggests that the MCD subtype is highly dependent on BCR signaling and downstream activation of the pro-survival NF-κB pathway. More than 80% of MCD tumors have mutations in the CD79B subunit of the BCR and/or mutations in the signaling adapter MYD88 (Ngo V N, et al. Oncogenically active MYD88 mutations in human lymphoma. Nature 2011; 470:115-9) that cooperatively activate NF-κB via the MYD88-TLR9-BCR (My-T-BCR) signaling complex (Phelan J D, et al. A multiprotein supercomplex controlling oncogenic signalling in lymphoma. Nature 2018; 560:387-91). These two mutations were associated with an 80% objective response rate in the phase II study of ibrutinib in relapsed/refractory DLBCL (Wilson W H, et al. Targeting B cell receptor signaling with ibrutinib in diffuse large B cell lymphoma. Nat Med 2015; 21:922-6).

While the initial analysis of the Phoenix clinical trial identified a survival benefit of ibrutinib addition to R-CHOP in younger patients, it did not assess whether specific genetic attributes of DLBCL tumors are associated with an improved survival in patients who received ibrutinib versus placebo (Younes A, et al., 2019). The present inventors tested the hypothesis that the classification of DLBCL into genetic subtypes could identify younger patients in Phoenix trial for whom ibrutinib provided a significant survival advantage, and discovered that the event-free and overall survival of subjects that are about 60 years of age or younger with certain genetic subtypes of DLBCL can be greatly improved by treatment with both ibrutinib and R-CHOP.

Accordingly, disclosed herein are methods for treating diffuse large B cell lymphoma comprising identifying a subject who (i) has diffuse large B cell lymphoma (DLBCL), (ii) exhibits an MCD genetic subtype or an N1 genetic subtype of DLBCL, and (iii) is 60 years of age or younger; and, treating the subject using a combination of a BTK inhibitor and R-CHOP chemotherapy. For purposes of clarity throughout the present disclosure, such methods may be referred to as “Method I”.

The identification of a subject who has DLBCL can use any acceptable method for DLBCL diagnosis. For example, diagnosis can be made from an excisional biopsy of a suspicious lymph node. Those of ordinary skill in the art readily appreciate other aspects of DLBCL diagnosis.

The identification of a subject who exhibits an MCD genetic subtype or an N1 genetic subtype of DLBCL preferably uses contemporary analysis of genetic aberrations that include mutations, copy number alterations, translocations, and optionally other relevant features. For example, the probabilistic predictor LymphGen can be used to assign an individual DLBCL tumor to one or more genetic subtypes, including MCD or N1. The LymphGen algorithm is described, for example, at Wright G W, et al. A Probabilistic Classification Tool for Genetic Subtypes of Diffuse Large B Cell Lymphoma with Therapeutic Implications. Cancer Cell 2020; 37:551-68 e14.

In accordance with the present methods, a subject who is 60 years of age or younger is identified. Previous work has determined that such subjects exhibit better event-free survival and overall survival rates using ibrutinib treatment, whereas older patients experience significantly more toxicity and lower survival. DLBCL patients tend to be middle-aged or older. In some embodiments, the subject is about 30, 35, 40, 45, 50, 55, or 60 years of age. For example, the subject may be about 40-60 years of age, or about 50-60 years of age. In some embodiments, the subject is about 50 years of age or younger.

The present inventors have discovered that treating the particular subset of subjects described herein using a combination of a BTK inhibitor and R-CHOP chemotherapy has a positive effect on the subjects' event-free survival and overall survival. For example, the treatment may result in an event-free survival rate of about 95-100% at about three years post-treatment. The treatment may also result in an event-free survival rate of about 95-100% at about four years post-treatment. The treatment may result in an overall survival rate of about 95-100% at about four years post-treatment. The treatment may also result in an overall survival rate of about 95-100% at about five years post-treatment.

The BTK inhibitor may be ibrutinib. In some embodiments, the BTK inhibitor is acalabrutinib. In other embodiments, the BTK inhibitor is zanabrutinib. The BTK inhibitor may be a single species or a combination of species. For example, the BTK inhibitor may be two or more of ibrutinib, acalabrutinib, or zanabrutinib.

Pursuant to the present methods, treatment with a BTK inhibitor may occur at substantially the same time as treatment with R-CHOP chemotherapy. Treatment with a BTK inhibitor that occurs at substantially the same time as R-CHOP chemotherapy refers to situations in which there is temporal overlap between the treatment with a BTK inhibitor and the R-CHOP chemotherapy. Accordingly, treatment with a BTK inhibitor that occurs during a time period that at least partially overlaps the time period during which R-CHOP chemotherapy occurs can be said to be at substantially the same time. In such instances, the treatment with a BTK inhibitor may commence before or after commencement of the R-CHOP chemotherapy. When there is no overlap between the time period during which treatment with a BTK inhibitor occurs and the time period during which R-CHOP chemotherapy occurs, then the treatments may be described as sequential. Thus, in certain embodiments, treatment with a BTK inhibitor and treatment with R-CHOP chemotherapy may occur sequentially. In such instances, the treatment with a BTK inhibitor may commence before or after commencement of the R-CHOP chemotherapy.

The specific characteristics of the BTK inhibitor therapy (e.g., dosage) may be in accordance with medically acceptable guidelines as are applicable to the specific subject being treated. Likewise, the specific characteristics of the R-CHOP chemotherapy (e.g., dosage) may be in accordance with medically acceptable guidelines as are applicable to the specific subject being treated.

The treatment with a BTK inhibitor and R-CHOP chemotherapy in accordance with the presently disclosed methods is not intended to be exclusive of other forms of therapeutic intervention on behalf of the subject, whether for DLBCL or for another condition that the subject is experiencing in addition to DLBCL.

Also disclosed are methods for treating diffuse large B cell lymphoma comprising identifying a subject who (i) has diffuse large B cell lymphoma (DLBCL), (ii) exhibits a set of genetic alterations characterized by: at least a MYD88L265P mutation and a CD79B mutation, or at least a MYD88L265P mutation and either a PIM1 or a BTG1 mutation, or at least a CD79B mutation and either a PIM1 or a BTG1 mutation, or at least a Notch1 truncating mutation, and, (iii) is 60 years of age or younger; and, treating the subject using a combination of a BTK inhibitor and R-CHOP chemotherapy.

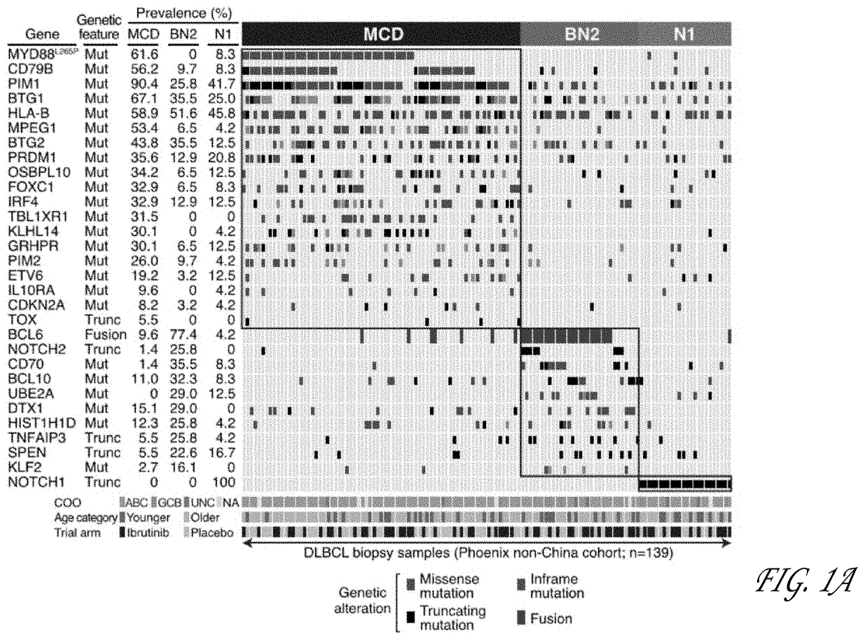

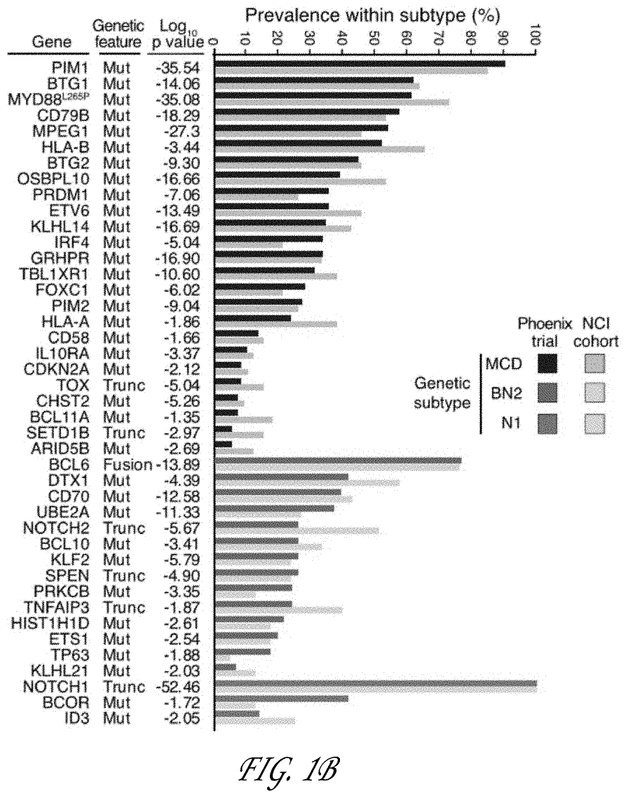

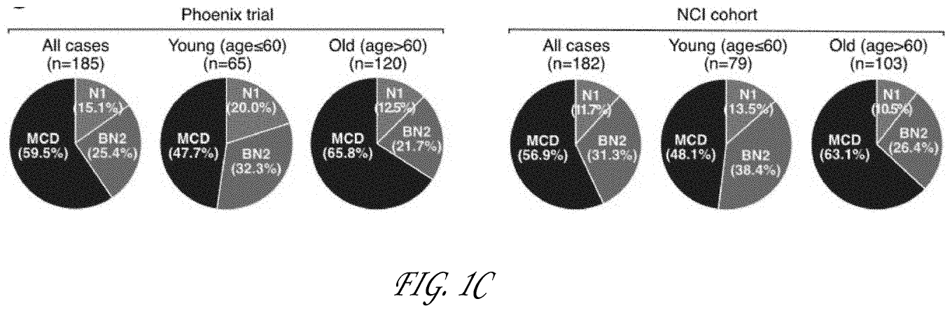

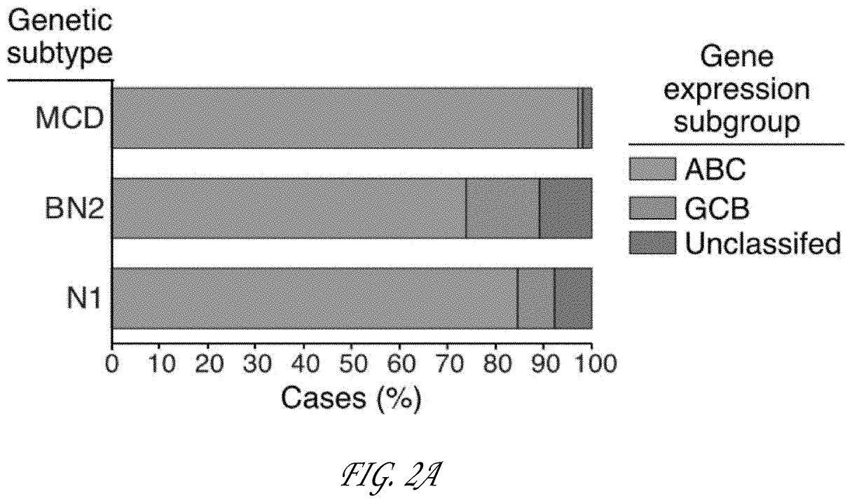

In some embodiments of such methods, the subject has a MYD88L265P mutation, a CD79B mutation, and either a PIM1 or a BTG1 mutation. As shown in FIGS. 1A and 1B, such mutations predominantly occur in subjects exhibiting an MCD genetic subtype of DLBCL. Other mutations that have been identified as being predominant in subjects exhibiting an MCD genetic subtype, including any of those disclosed in FIGS. 1A and 1B, can be used in order to identify a subject for treatment with both a BTK inhibitor and R-CHOP chemotherapy in accordance with the present methods. In certain embodiments, the subject has a MYD88L265P mutation, a CD79B mutation, and both a PIM1 and a BTG1 mutation.

In certain embodiments of the present methods, the subject exhibits at least a Notch1 truncation. As shown in FIGS. 1A and 1B, such genetic aberrations predominantly occur in subjects exhibiting an N1 genetic subtype of DLBCL. Other mutations that have been identified as being predominant in subjects exhibiting an N1 genetic subtype, including any of those disclosed in FIGS. 1A and 1B, can be used in order to identify a subject for treatment with both a BTK inhibitor and R-CHOP chemotherapy in accordance with the present methods.

The other characteristics of the present methods may be in accordance with any of the embodiments described supra in connection with Method I.

### EXAMPLES

The present invention is further defined in the following Examples. It should be understood that these examples, while indicating preferred embodiments of the invention, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and conditions.

### Example 1—Phenotyping of DLBCL Genetic Subtypes

DLBCL tumors were obtained from 773 patients on the Phoenix clinical trial (Younes A, et al. 2019), including 189 enrolled in China (“China” cohort) and 584 enrolled elsewhere (“non-China” cohort). Formalin-fixed and paraffin-embedded (FFPE) biopsies samples were subjected to whole exome sequencing (China cohort) or were analyzed by targeted resequencing of 99 genes that are recurrently mutated genes in lymphoma (Nugen) as well as by RNA sequencing (non-China cohort). Based on the genomic aberrations detected in these biopsies, the LymphGen algorithm was used to assign tumors to three genetic subtypes that are prevalent in non-GCB DLBCL: MCD, BN2 and N1. Some unassigned biopsies likely belong to the A53 genetic subtype, which is also prevalent in non-GCB DLBCL but could not be identified in the absence of DNA copy number data. As previously observed, some unassigned biopsies were genetically composite, bearing mutations characteristic of more than one subtype, while others lacked sufficient subtype-defining mutations to be classified.

FIG. 1A illustrates the distribution and prevalence of genetic aberrations in DLBCL genetic subtypes in the Phoenix non-China cohort. Missense or inframe deletion/insertion mutations (Mut), protein-truncating mutations (Trunc), and gene rearrangement (Fusion) are shown as indicated. Also shown is the cell-of-origin (COO) gene expression subgroup (ABC: ABC DLBCL, GCB: GCB DLBCL, UNC: Unclassified DLBCL, NA: Not available), the age category (Younger: <60, Older: >60), and the Phoenix study arm. FIG. 1B shows the prevalence and significance of association between genetic aberrations and the DLBCL genetic subtypes. P values compare each subtype to all other cases in the Phoenix set of cases. The prevalence of each genetic aberration in non-GCB biopsies from the NCI cohort is shown for comparison. FIG. 1C illustrates the distribution of the genetic subtypes among younger (age≤60), older (age>60) and all patients in the Phoenix and NCI cohorts.

Overall, 185 (23%) Phoenix biopsies were assigned to a single genetic subtype using a high confidence threshold (≥90%). The genetic features of the identified subtypes—MCD, BN2 and N1¬significantly distinguished each from all other DLBCLs from the Phoenix trial (p<0.01) and occurred with frequencies that mirror those in non-GCB DBLCL cases in an observational “NCI cohort” 4,5 (FIG. 1A and FIG. 1B). The subtype-defining genetic lesions identified in the China and non-China Phoenix cohorts were comparable, further demonstrating the reproducibility of the genetic subtype assignment. The MCD subtype accounted for the most cases (n=110, 59.5%) followed by BN2 (n=47, 25.4%) and N1 (n=28, 15.1%), a distribution similar to that observed in the NCI cohort (FIG. 1C). The subtype distributions in the China and non-China cohorts were also comparable, as were the distributions among younger (age≤60) and older (age>60) patients.

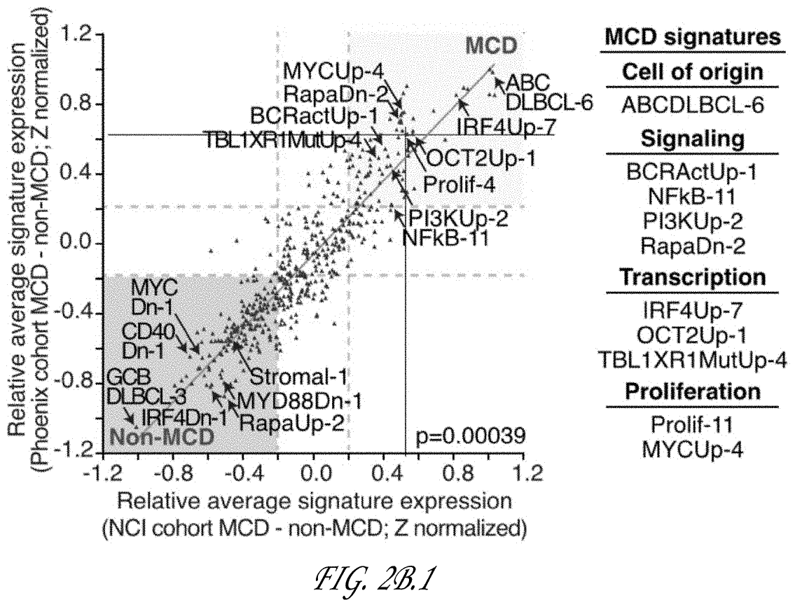

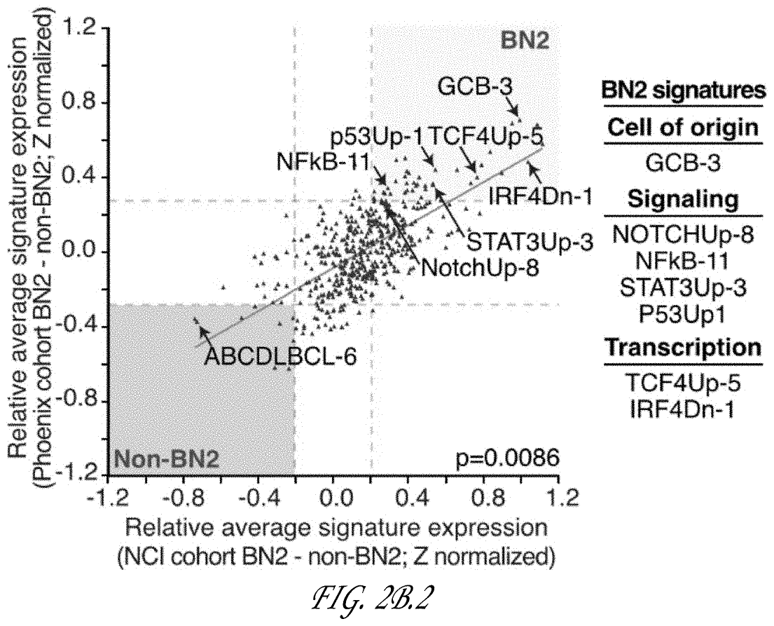

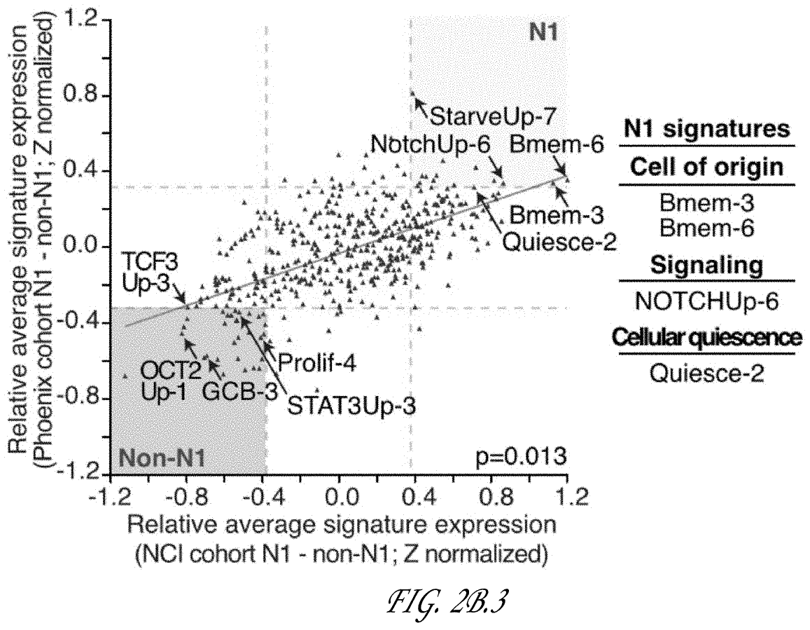

Phenotypes of DLBCL genetic subtypes. RNA sequencing data from the non-China cohort were used to assess tumor phenotypes in each genetic subtype. FIG. 2A illustrates the distribution of DLBCL gene expression subgroups among cases assigned to the MCD, BN2 and N1 genetic subtypes. FIGS. 2B.1, 2B.2, and 2B.3 show a comparison of gene expression signature averages between genetic subtypes and all other DLBCL samples. Shown are Z scores (see Methods) representing the difference between the mean expression values of each gene expression signature between samples assigned to the indicated genetic subtype and all other DLBCLs. Z scores calculated for the Phoenix and NCI cohorts are plotted on the y-axis and x-axis, respectively. The brown dashed lines indicate the Z scores corresponding to a significant difference between the two sample subsets (p=0.01). The yellow shaded areas are those signatures more highly expressed in the indicated subtype relative to other samples and the blue shaded areas are signatures that are significantly under-expressed in the indicated subtype relative to all other samples. The indicated p value is for the correlation of the Z scores of each signature within the two cohorts. FIG. 2C illustrates extranodal involvement in the genetic subtypes, subdivided by anatomic site as indicated. P values are from a 2-way Fisher's Exact test. ns: non-significant.

As reported (Wright G W, et al., Cancer Cell 2020), most MCD tumors had an ABC gene expression profile (p=5.4E-5) while N1 and especially BN2 had a higher percentage of GCB and Unclassified cases (Table 1; FIG. 2A).

TABLE 1

| | | | | | | | | | | | | | | Subtype |
|---|---|---|---|---|---|---|---|---|---|---|---|---|---|---|
| | | | | | MCD subtype | | | BN2 subtype | | | N1 subtype | | | P value |
| | | Total | Young | Old | Total | Young | Old | Total | Young | Old | Total | Young | Old | Total |
| Cell of | Age (average) | 59.8 | 48.1 | 69.0 | 64.1 | 51.7 | 68.9 | 61.3 | 52.1 | 68.7 | 59.6 | 46.5 | 71.0 | 0.12* |
| origin | Gender (% male) | 58.4 | 44.6 | 65.8 | 59.6 | 38.7 | 67.1 | 59.1 | 52.4 | 65.4 | 53.6 | 46.2 | 60.0 | 0.87^{†} |
| | ABC (%) | 76.6 | 70.2 | 81.5 | 97.1 | 90.0 | 100.0 | 73.9 | 71.4 | 76.0 | 84.6 | 90.9 | 80.0 | 0.000053^{†} |
| | GCB (%) | 16.8 | 21.4 | 13.3 | 1.0 | 3.3 | 0.0 | 15.2 | 19.0 | 12.0 | 7.7 | 0.0 | 13.3 | 0.00037^{†} |
| | Unclass (%) | 6.6 | 8.4 | 5.3 | 1.9 | 6.7 | 0.0 | 10.9 | 9.5 | 12.0 | 7.7 | 9.1 | 6.7 | 0.034^{†} |
| International | IPI (average) | 2.3 | 1.9 | 2.7 | 2.5 | 2.3 | 2.6 | 2.4 | 2.0 | 2.7 | 2.5 | 1.9 | 2.9 | 0.83^{†} |
| Prognostic | IPI sans age | 1.8 | 1.9 | 1.7 | 1.8 | 2.3 | 1.6 | 1.9 | 2.0 | 1.7 | 1.9 | 1.9 | 1.9 | 0.79* |
| Index | (average) | | | | | | | | | | | | | |
| | IPI 0-1 (%) | 24.3 | 37.6 | 13.9 | 16.4 | 19.4 | 15.2 | 17.0 | 28.6 | 7.7 | 25.0 | 38.5 | 13.3 | 0.53^{†} |
| | IPI 2-3 (%) | 62.2 | 58.8 | 64.9 | 67.3 | 67.7 | 67.1 | 70.2 | 71.4 | 69.2 | 53.6 | 46.2 | 60.0 | 0.32^{†} |
| | IPI 4-5 (%) | 13.5 | 3.5 | 21.2 | 16.4 | 12.9 | 17.7 | 12.8 | 0.0 | 23.1 | 21.4 | 15.4 | 26.7 | 0.58^{†} |
| | Age > 60 (%) | 56.0 | 0.0 | 100.0 | 71.8 | 0.0 | 100.0 | 55.3 | 0.0 | 100.0 | 53.6 | 0.0 | 100.0 | 0.052^{†} |
| | ECOG performance | 12.0 | 14.1 | 10.4 | 10.0 | 29.0 | 2.5 | 14.9 | 14.3 | 15.4 | 28.6 | 30.8 | 26.7 | 0.051^{†} |
| | status > 1 (%) | | | | | | | | | | | | | |
| | LDH > 1 (%) | 54.9 | 60.9 | 50.1 | 53.6 | 71.0 | 46.8 | 63.8 | 76.2 | 53.8 | 64.3 | 61.5 | 66.7 | 0.39^{†} |
| | Extranodal sites > | 33.8 | 36.5 | 31.6 | 40.9 | 48.4 | 38.0 | 23.4 | 28.6 | 19.2 | 17.9 | 15.4 | 20.0 | 0.020^{†} |
| | 1 (%) | | | | | | | | | | | | | |
| | Stage > 2 (%) | 76.5 | 77.4 | 75.8 | 74.5 | 80.6 | 72.2 | 83.0 | 81.0 | 84.6 | 82.1 | 84.6 | 80.0 | 0.47^{†} |

*3-way P test
^{†}3-way Fisher's Exact Test

Next, for each case, the expression of gene signatures were calculated that reflect biological attributes of normal lymphocytes and lymphoid malignancies (Schmitz R, et al. 2018; Wright G W, 2020; Shaffer A L, et al. A library of gene expression signatures to illuminate normal and pathological lymphoid biology. Immunological reviews 2006; 210:67-85). To compare the genetic subtypes in the Phoenix trial with those in the NCI cohort, the average expression of each gene signature in each subtype was computed. Signature expression levels in the two cohorts were significantly correlated for the MCD (p=0.00039), BN2 (p=0.0086), and N1 (p=0.013) subtypes (FIGS. 2B.1, 2B.2, 2B.3).

MCD was characterized by signatures of oncogenic BCR, NF-kB and PI3 kinase signaling, signatures of subtype-defining transcription factors (IRF4, TBL1XR1, Oct-2), and signatures of cellular proliferation. BN2 expressed a signature of Notch pathway activation as well as signatures of NF-kB, STAT3 and p53 activity. N1 also expressed a Notch activation signature, as well as signatures of cellular quiescence (low proliferation) and the memory B-cell differentiation state.

Clinical attributes of DLBCL genetic subtypes. MCD had a higher proportion of older patients than BN2 and N1 (p=0.019), a distribution mirrored in the NCI cohort (Table 1; FIG. 1C). The Phoenix MCD patients had more frequent extranodal involvement than the other subtypes (p=0.0066; FIG. 2C), as in the NCI cohort (Wright G W, et al. 2020). Previous genetic analysis revealed a striking similarity between MCD and a specific subset of primary extranodal lymphomas, including those that occur the central nervous system (CNS), testis, breast, adrenal, ovary, and uterus (Id.). Involvement of these primary extranodal sites was significantly more frequent in MCD than in BN2 or N1 (p=0.018), with the exception of CNS involvements, which was an exclusion criterion for the Phoenix trial. Among cases with extranodal disease, these primary extranodal sites were involved significantly more often in MCD (32.1%) than in BN2 (17.8%) and N1 (9.5%) cases (p=0.042).

Overall, the genetic subtypes had similar proportions of cases with low (0-1), intermediate (2-3) and high (4-5) values of the International Prognostic Index (IPI) (Table 1). Among individual IPI variables, the subtypes differed with respect to extranodal involvement, as mentioned above, and ECOG performance status, which was more frequently adverse in N1 than in BN2 and MCD (P=0.033). Younger MCD patients had a higher age-adjusted IPI than older patients (2.3 vs. 1.6, respectively; p=0.0018), which was driven by poor ECOG scores and elevated LDH among younger patients. IPI was not significantly different between younger and older BN2 and N1 patients.

### Example 2—Effect of Ibrutinib In DLBCL Genetic Subtypes

Following the genetic subtyping described in Example 1, it was investigated whether the addition of ibrutinib to R-CHOP chemotherapy was associated with an improved outcome in genetic subtypes of DLBCL. With a median follow-up of 46.2 months, the 3-year event-free survival (EFS) estimates were 65% for MCD, 74% for BN2 and 55% for N1 for all patients treated on the R-CHOP only arm. While these outcomes are better than for R-CHOP-treated patients in the NCI cohort, they may reflect the known accrual bias toward favorable prognosis in phase III lymphoma trials (Maurer M J, et al. Diagnosis-to-Treatment Interval Is an Important Clinical Factor in Newly Diagnosed Diffuse Large B-Cell Lymphoma and Has Implication for Bias in Clinical Trials. J Clin Oncol 2018; 36:1603-10). For patients treated on the ibrutinib arm, the present inventors separately analyzed EFS and overall survival (OS) by age because of the increased toxicity observed in patients older than 60 years, an effect not observed on the R-CHOP arm (Younes, et al., 2019). As reported, older patients did not benefit from ibrutinib, and the inventors' analysis shows this is regardless of genetic subtype.

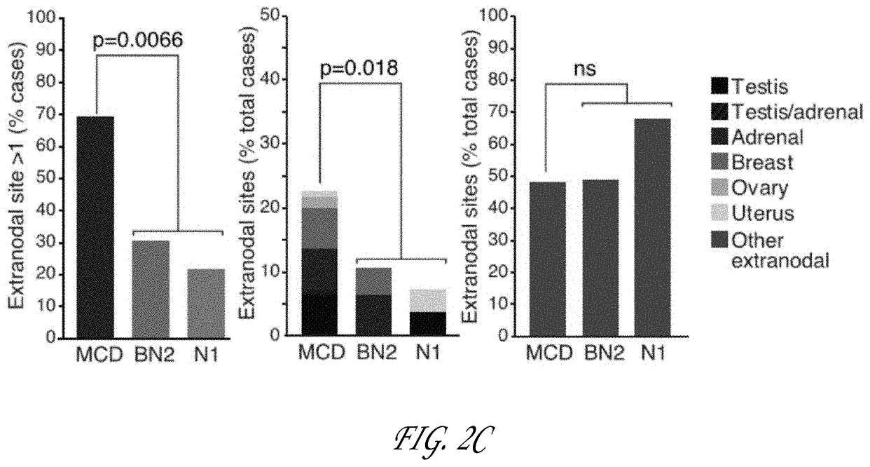

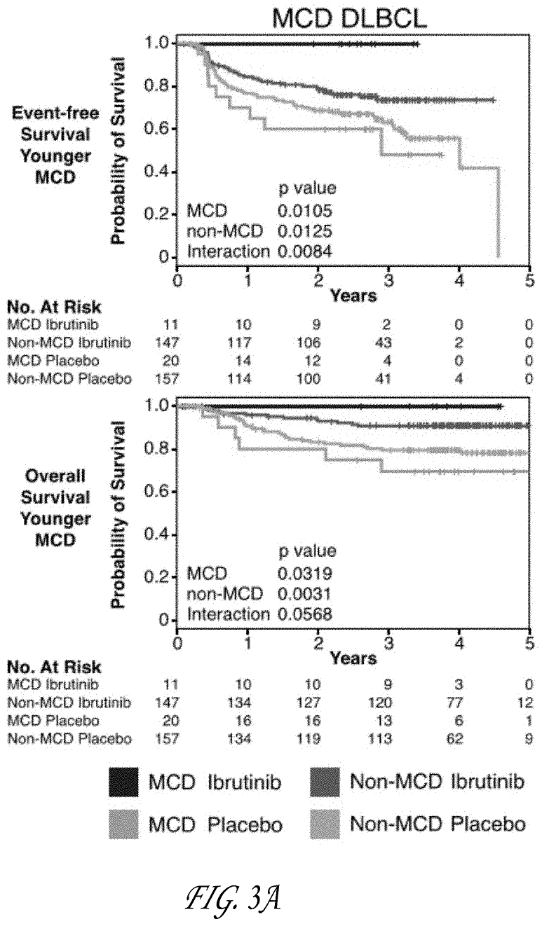

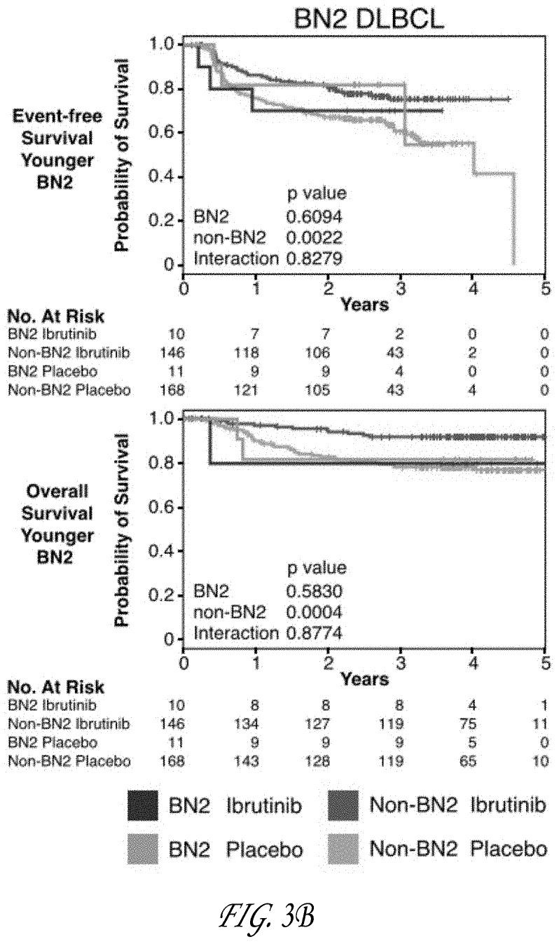

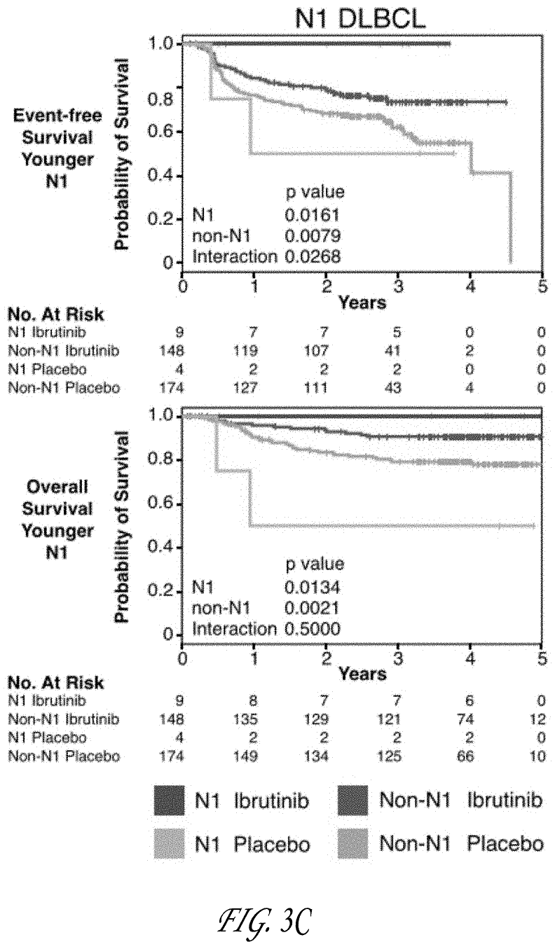

FIGS. 3A-3C provide Kaplan-Meier plots of event-free and overall survival in younger (age≤60) and older (age>60) patients assigned to the MCD, BN2 and N1 genetic subtypes. Shown are log-rank p values for the difference in survival in the indicated genetic subtype treated with R-CHOP plus ibrutinib or placebo. The interaction p value indicates the significance of the difference in ibrutinib benefit within the indicated genetic subtype compared with all other DLBCLs.

Younger patients with MCD DLBCL (n=31) who received ibrutinib and R-CHOP had an EFS and OS of 100% at 3-years, compared to a significantly lower EFS of 48% (p=0.01) and OS of 69.6% (p=0.032) when treated with R-CHOP alone. Although younger non-MCD patients also benefitted from ibrutinib, the effect was greater in those with MCD, with interaction p values for EFS and OS of 0.0084 and 0.057, respectively (FIGS. 3A-3C). The survival advantage conferred by ibrutinib was apparent in both the China and non-China cohorts, albeit with less statistical significance due to smaller sample sizes, demonstrating the reproducibility of this effect.

Ibrutinib addition was also associated with improved survival in younger patients with N1 DLBCL (n=13). These N1 patients had a 3-year EFS and OS of 100% whereas those treated with R-CHOP alone had a significantly inferior EFS (50%; p=0.0161) and OS (50%; p=0.0134). Ibrutinib had a greater effect on EFS in younger N1 patients than in non-N1 patients (interaction p value=0.0268). By contrast, ibrutinib did not benefit younger patients with BN2 DLBCL (n=21) (FIGS. 3A-3C).

### Example 3—Genetic and Phenotypic Attributes of N1 DLBCL

The benefit of ibrutinib in N1 was unanticipated based on previous analysis of its biological attributes (Schmitz R, et al., 2019), which was hampered by a small sample size (n=16). To elucidate the molecular basis for ibrutinib efficacy in N1 patients, publicly available tumor sequencing data for NOTCH1 mutations were analyzed, such mutations being necessary and sufficient for N1 classification.

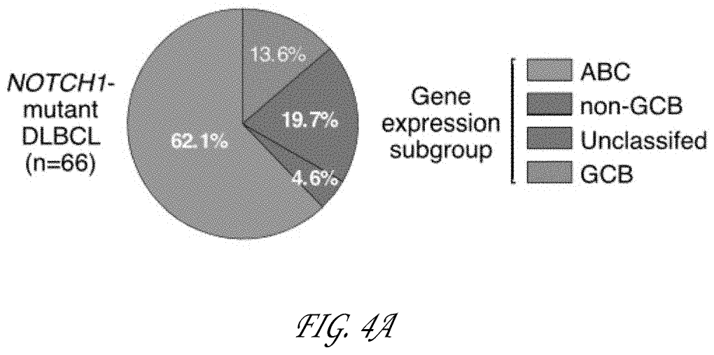

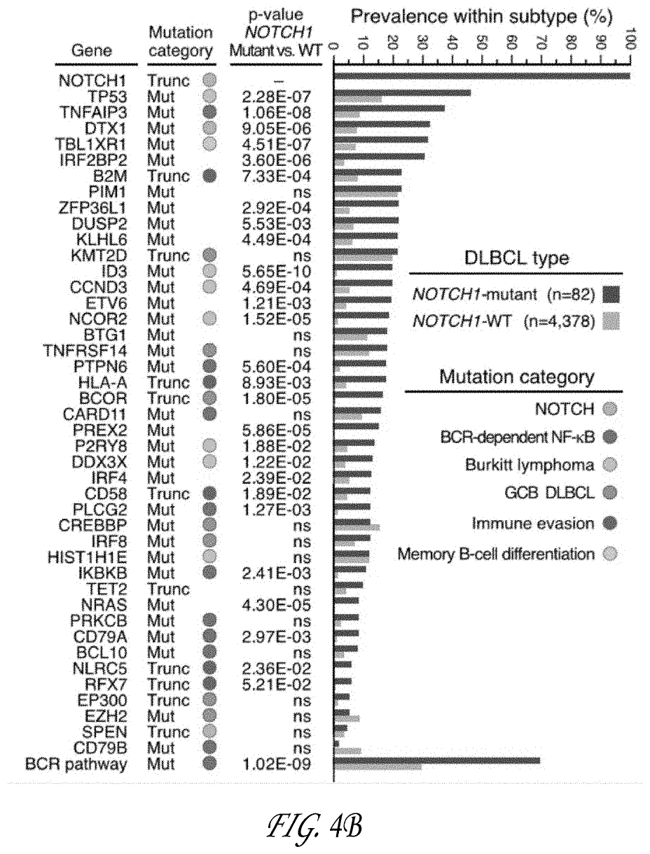

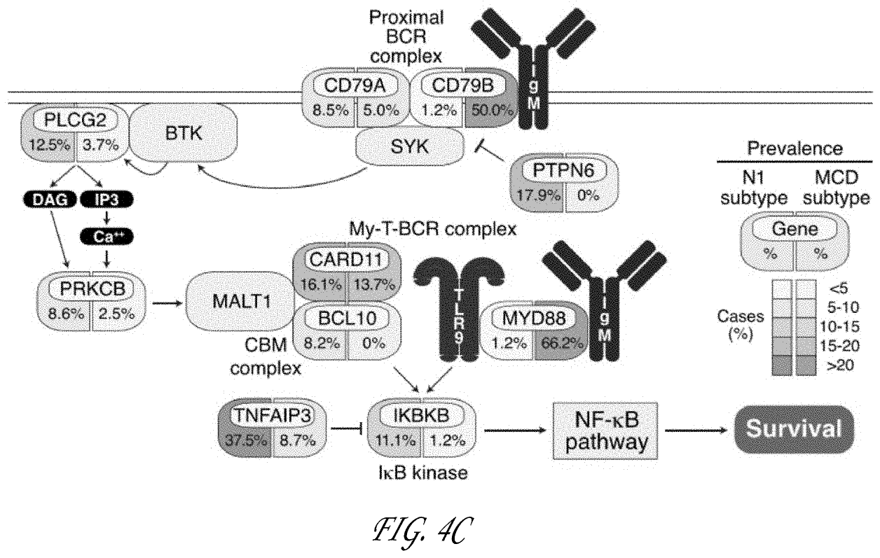

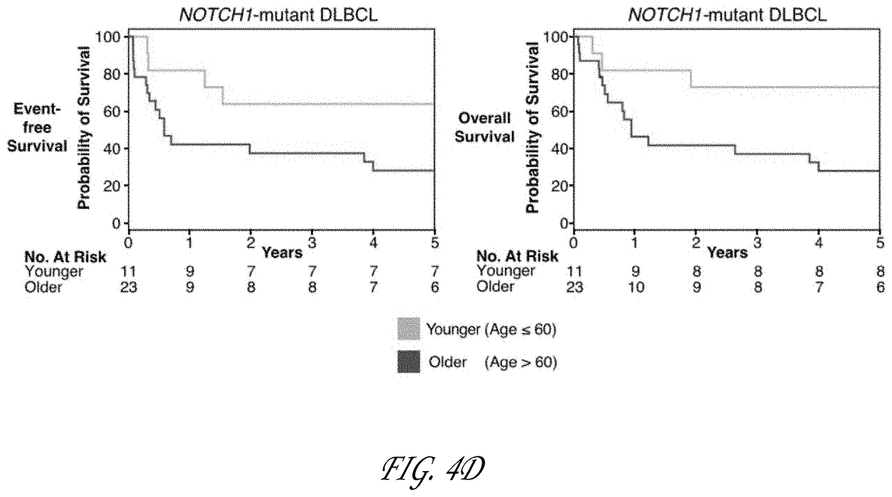

FIG. 4A shows the distribution of DLBCL gene expression subgroups among NOTCH1-mutant DLBCLs. FIG. 4B shows recurrently mutated genes in NOTCH1-mutant DLBCL. Shown are type and prevalence of mutations in the indicated genes among NOTCH1-mutant and NOTCH1 wild type DLBCLs. Genes are assigned to functional categories, as indicated. Mut: Non-synonymous mutation; Trunc: Truncating mutation; WT: wild type; ns: non-significant FIG. 4C provides a schematic of the BCR-dependent NF-κB pathway showing the prevalence of mutations targeting each pathway component in the N1 (NOTCH1-mutant) and MCD subtypes of DLBCL according to the color scale shown. DAG: diacylglyerol; IP3: inositol triphosphate; Ca++: calcium ion; CBM complex: CARD11-BCL10-MALT1 complex; My-T-BCR: MYD88-TLR9-B cell receptor complex. FIG. 4D provides Kaplan-Meier plots of event-free and overall survival in younger (age≤60) and older (age>60) patients with NOTCH1-mutant DLBCL treated with R-CHOP-like chemotherapy, curated from the published literature

From 4,460 DLBCL cases, 82 (1.8%) NOTCH1-mutant cases were identified (NOTCH1-mutant DLBCL cases were ascertained from a compilation of whole exome, whole genome, transcriptome and targeted DNA resequencing data from 5,754 cases of nodal DLBCL, curated from 33 studies). The cell-of-origin assignment of these cases, when available (n=66), was primarily non-GCB (86.4%) (FIG. 4A).

The present inventors next identified genes that were recurrently mutated (>5% of cases) in this larger cohort of N1 cases, 92.5% (37/40) of which were mutated more frequently than in NOTCH1 wild type (WT) cases (FIG. 4B). These genes were grouped into functional categories that revealed new aspects of N1 biology (FIG. 4B). N1 mutations frequently inactivated SPEN, a negative regulator of NOTCH1 signaling, and were also recurrent in DTX1, a NOTCH1 transcriptional target. Given the non-GCB phenotype of most N1 tumors, it was surprising to observe mutations in genes implicated in the pathogenesis of GCB DLBCL and Burkitt lymphoma, which are both derived from germinal center B-cells (FIG. 4B). Unlike GCB DLBCL and Burkitt lymphoma, however, N1 frequently acquired TBL1XR1 mutations that promote memory B-cell differentiation (Venturutti L, et al. TBL1XR1 Mutations Drive Extranodal Lymphoma by Inducing a Pro-tumorigenic Memory Fate. Cell 2020; 182:297-316 e27), befitting the memory B-cell phenotype of this subtype (FIGS. 2B.1, 2B.2, 2B.3).

A second biological theme was evasion of immune surveillance: N1 recurrently inactivated β2-microglobulin, HLA-A, and transactivators of MHC class I expression (NLRC5, RFX7), thereby hindering antigen presentation, and also inactivated CD58, which is required for NK cell activation.

Most notably, from the perspective of the Phoenix trial, N1 mutations recurrently targeted components of the BCR-dependent NF-κB pathway, including the CD79A subunit of the BCR itself (FIGS. 4B, 4C). Altogether, 69.6% of N1 tumors harbored BCR pathway mutations, which was significantly greater than in NOTCH1-WT DLBCLs (29.6%, p=1.02E-9). Unlike MCD tumors, which primarily engage the BCR pathway by acquiring CD79B and MYD88 mutations, N1 mutations preferentially target mediators and regulators of BCR signaling, including PTPN6 (SHP-1), Phospholipase-Cy2 (PLCG2), BCL10, CARD11, IκB kinase (IKBKB), and A20 (TNFAIP3) (FIG. 4C).

Given the limited number of younger N1 patients on the Phoenix placebo arm, the present inventors endeavored to provide further evidence that R-CHOP alone is insufficient to achieve exceptional EFS and OS in N1. Among 34 younger patients treated with R-CHOP-like regimens from the literature-curated N1 cohort, 3-year EFS and OS were 64% and 73%, respectively, consistent with the survival of comparable Phoenix trial patients (FIG. 4E).

Accordingly, provided herein is a precision medicine framework for the use of BTK inhibitors in previously untreated younger patients with non-GCB DLBCL. The inventors identified two genetic subtypes of DLBCL—MCD and N1—for which the addition of ibrutinib to R-CHOP chemotherapy improved survival. More broadly, ibrutinib was also beneficial in younger patients not classified as MCD or N1, albeit to a lesser degree, suggesting that the utility of BTK inhibitors may extend to other patients with non-GCB DLBCL as well.

The starting hypothesis was that patients with MCD DLBCL would benefit from the addition of ibrutinib to chemotherapy. The chronic active BCR signaling in MCD renders the tumor cells highly dependent on BTK to activate the pro-survival NF-κB pathway (Davis R E, et al. Chronic active B-cell-receptor signaling in diffuse large B-cell lymphoma. Nature 2010; 463:88-92). As a result, patients with MCD tumors frequently respond to ibrutinib monotherapy, as do patients with primary central nervous system lymphoma (Lionakis M S, et al. Inhibition of B Cell Receptor Signaling by Ibrutinib in Primary CNS Lymphoma. Cancer Cell 2017; 31:833-43 e5), which bears the MCD genotype (id; Wright, et al., 2020). In addition, the inhibition of NF-κB by ibrutinib is likely to sensitize MCD tumors to R-CHOP given the ability of NF-κB to suppress the apoptotic response to cytotoxic chemotherapy (Wang, C Y, et al., NF-kappaB antiapoptosis: induction of TRAF1 and TRAF2 and c-IAP1 and c-IAP2 to suppress caspase-8 activation. Science 1998; 281:1680-3), These considerations together with the inventors' analysis of the Phoenix trial firmly establish the rationale for ibrutinib use in the treatment of MCD-like lymphomas.

The sensitivity of N1 DLBCL to ibrutinib plus R-CHOP therapy was unanticipated, largely because the previous analysis of N1 was limited by its relative rarity. The biology of N1 DLBCL came into focus from our genetic analysis of a larger cohort of NOTCH1-mutant tumors gleaned from the published literature. Many N1 mutations suggest a germinal center origin, but it adopts a memory B-cell phenotype, in part due to frequent TBL1XR1 mutations that foster memory B-cell differentiation (Venturutti L, et al., 2020). Importantly, N1 DLBCLs engage the BCR-dependent NF-κB pathway by diverse mechanisms in a majority of cases (˜70%), including CD79A mutations, which enhance proximal BCR signaling by increasing cell surface BCR expression (Wilson W H, et al., 2015).

The present disclosure represents an important outgrowth of the recent genetic classification system of DLBCL in a clinical trial setting and supports its utility in defining pathogenetically related tumors that respond similarly to therapy. The accurate classification of tumors using the LymphGen algorithm was evidenced by the consistent association of DLBCL genetic subtypes with particular tumor phenotypes, as judged by gene expression profiling, and by clinical attributes, such as the frequent extranodal involvement of MCD tumors. The present classification was performed using sequencing data from FFPE tumors, showing that it can be used to elucidate therapeutic responses in clinical trials, which typically collect FFPE biopsies.

By providing a mechanistic basis for the survival benefit of ibrutinib addition to R-CHOP in younger DLBCL patients, the present disclosure casts the Phoenix trial in a new light. The effect of patient age on response to ibrutinib was a secondary, not primary, endpoint of the Phoenix trial, and as such was considered a subset analysis. Nonetheless, the present discovery that molecular subsets of younger patients responded preferentially to ibrutinib indicates that the overall significant benefit of ibrutinib in this age group should not be discounted as a statistical aberration. The failure of ibrutinib to improve survival in older patients can be explained by the added toxicity of ibrutinib in this age group, which often prevented the full administration of chemotherapy. Since the addition of ibrutinib to R-CHOP did not significantly increase toxicity in younger patients with non-GCB DLBCL, it is rational to consider this therapeutic combination for such patients.

What is claimed:

1. A method for treating diffuse large B cell lymphoma comprising:
identifying a subject who (i) has diffuse large B cell lymphoma (DLBCL), (ii) exhibits an MCD genetic subtype or an N1 genetic subtype of DLBCL, and (iii) is 60 years of age or younger; and,
treating the subject using a combination of a BTK inhibitor and R-CHOP chemotherapy.

2. The method according to claim 1, wherein the subject is about 60 years of age.

3. The method according to claim 1, wherein the subject is about 40-60 years of age.

4. The method according to claim 1, wherein the subject is about 50-60 years of age.

5. The method according to claim 1, wherein the subject is about 50 years of age or younger.

6. The method according to claim 1, wherein the subject exhibits an MCD genetic subtype.

7. The method according to claim 1, wherein the subject exhibits an N1 genetic subtype.

8. The method according to claim 1, wherein the treatment results in an event-free survival rate of about 95-100% at about three years post-treatment.

9. The method according to claim 1, wherein the treatment results in an event-free survival rate of about 95-100% at about four years post-treatment.

10. The method according to claim 1, wherein the treatment results in an overall survival rate of about 95-100% at about four years post-treatment.

11. The method according to claim 1, wherein the treatment results in an overall survival rate of about 95-100% at about five years post-treatment.

12. The method according to claim 1, wherein treatment with a BTK inhibitor occurs at substantially the same time as treatment with R-CHOP chemotherapy.

13. The method according to claim 1, wherein treatment with a BTK inhibitor and treatment with R-CHOP chemotherapy occur sequentially.

14. The method according to claim 1, wherein the BTK inhibitor is ibrutinib.

15. The method according to claim 1, wherein the BTK inhibitor is acalabrutinib.

16. The method according to claim 1, wherein the BTK inhibitor is zanabrutinib.

17. A method for treating diffuse large B cell lymphoma comprising:
identifying a subject who
(i) has diffuse large B cell lymphoma (DLBCL), (ii) exhibits a set of genetic alterations characterized by:
at least a MYD88L265P mutation and a CD79B mutation,
or at least a MYD88L265P mutation and either a PIM1 or a BTG1 mutation, or at least a CD79B mutation and either a PIM1 or a BTG1 mutation,
or at least a Notch1 truncating mutation,
and, (iii) is 60 years of age or younger; and,
treating the subject using a combination of a BTK inhibitor and R-CHOP chemotherapy.
